(19) United States
(12) Patent Application Publication
(10) Pub. No.: US 2025/0354987 A1
(43) Pub. Date: Nov. 20, 2025

(54) SYSTEMS AND METHODS FOR QUANTITATIVE LATERAL FLOW TESTS

(71) Applicant: TALARA BIO INC., Kirkland (CA)

(72) Inventors: Brian DOYLE, Kirkland (CA); Pablo MENGHINI, Kirkland (CA); Yu GUAN, Kirkland (CA)

(21) Appl. No.: 19/210,228

(22) Filed: May 16, 2025

Related U.S. Application Data
(60) Provisional application No. 63/649,117, filed on May 17, 2024.

(51) Int. Cl. G01N 33/543; B01L 3/00; G01N 21/47; G16H 10/40

(52) U.S. Cl. CPC G01N 33/54388; B01L 3/5023; G01N 21/4738; G16H 10/40; B01L 2300/0825; B01L 2300/0887; G01N 2021/4771

(57) ABSTRACT

A method for determining a target analyte level comprising: obtaining test data of a test region of a lateral flow device, test data comprising test data values of a light parameter of light reflected from test region; dividing test data into test data sub-groups; obtaining light control data of a light control region, light control data comprising light data values of the light parameter from the light control region; dividing light control data into light control data sub-groups, each light control data sub-group having a corresponding test data sub-group; for at least some of the test data sub-groups, comparing test data values of a given test data sub-group with light data values of the corresponding light control data sub-group, and correcting, if any variations are determined, test data values; and comparing corrected test data with predetermined correlation data correlating light data values with different target analyte levels.

### CROSS-REFERENCE TO RELATED APPLICATIONS

This application claims the benefit of U.S. Provisional Patent Application Ser. No. 63/649,117, filed on May 17, 2024. The contents of the aforementioned application are incorporated by reference herein.

### FIELD

The present technology relates to systems and methods for quantitative lateral flow tests.

### BACKGROUND

Lateral flow tests are used for diagnosis and broadly comprise a lateral flow assay performed by a lateral flow device for determining a concentration of a compound in a sample. Many different lateral flow tests exist for determining different types of compounds. Lateral flow tests can be performed at the point-of-care, such as at home, are relatively inexpensive and provide relatively quick results. Example uses of lateral flow tests include those for detecting pregnancy from a urine sample, and those for detecting COVID-19 infection from a nasal/throat swab. However, existing lateral flow tests suffer from the limitation of only being able to provide qualitative or semi-quantitative test results. Quantitative test results could be vital in monitoring a progress of a disease or infection, for example.

In some lateral flow tests, the compound of interest is an antigen, the underlying technology is an immunographic assay, and the lateral flow device is a cassette housing a test strip on which the immunographic assay is performed. The test strip is configured to cause a fluid biological sample of the user (e.g. urine, saliva, blood, plasma) applied thereto to move along the test strip by capillary action. As the fluid moves along the test strip it will encounter reporter molecules (such as gold linked to an antibody, aptamer or detecting molecule). Target analyte in the sample will bind with the reporter molecules and migrate along the strip to test and control lines where differential binding results in a change of color.

The results of lateral flow tests are typically determined by a visual reading of the test band. Theoretically, an intensity of the test band could be used to semi-quantitatively or quantitatively determine the concentration of the compound of interest in the sample as there is a link between the visual intensity and concentration of the compound of interest. However, the intensity of the markings cannot be easily determined in a reproducible and consistent manner. Therefore, conventional lateral flow test devices only provide qualitative or semi-quantitative results. Other types of lateral flow test readers typically require readers.

It is an object of the present technology to ameliorate at least some of the limitations present in the prior art.

### SUMMARY

Embodiments of the present technology have been developed based on developers' appreciation of certain shortcomings associated with existing lateral flow devices. Existing lateral flow devices provide qualitative data in the form of a test marker such as a visual band appearing on a test strip after contact with a sample.

Specialized reading devices exist which are designed to “read” the test band and output a semi-quantitative reading based on an intensity of the test band. However, such specialised reading devices are expensive and inconvenient for home users. Other systems have attempted to use the user's own smart device for imaging the test band and determining a quantitative or quantitative reading of the test band based on the detected intensity. However, these approaches are also inconsistent and unreliable. Developers have determined that at least some of the inconsistencies may be due to varying ambient light conditions which affect the detected visual parameters of the test band.

Broadly, aspects and embodiments of the present technology comprise systems and methods for determining quantitative test results from lateral flow devices. In certain embodiments, such systems and methods provide reliable and reproducible results regardless of the ambient light conditions. In certain embodiments, the methods can be executed by a smart device of the user meaning that specialised devices are not required, rendering such methods widely accessible.

Other aspects of the current technology are directed to improved lateral flow test devices and their methods of manufacture.

Developers have developed markings for a test cassette and associated computer-implemented method that can “read” the markings as well as the test indicator and convert the visual test indicator to an intensity of the positive or negative test result. The markings may be attached to the test cassette or formed therewith. Broadly, the method can correct for shadows and other visual anomalies on the visual test indicator rendering more accurate analyte level results.

From some aspects, there is provided a method for determining a level of a target analyte in a sample from a lateral flow device, the method being executed by a processor of a computer system, the method comprising: obtaining test data of a test region of a lateral flow device to which the sample has been applied, the test data of the test region comprising test data values of at least one light parameter of light reflected from the test region; dividing the test data into test data sub-groups based on subdivided areas of the test region; obtaining light control data of a light control region of the lateral flow device, the light control data comprising light data values of the at least one light parameter of light reflected from the light control region; dividing the light control data into light control data sub-groups based on subdivided areas of the light control region, each light control data sub-group having a corresponding test data sub-group; for at least some of the test data sub-groups, comparing the test data values of a given test data sub-group with the light data values of the corresponding light control data sub-group, and correcting, if any variations are determined between the test data values of the given test data sub-group with the light data values of the corresponding light control data sub-group, the test data values of the given test data sub-group; and determining the level of the target analyte of the test data by comparing the corrected test data with predetermined correlation data correlating light data values with different target analyte levels.

In certain embodiments, the correcting comprises adjusting the test data values only of the given test data sub-group for which variation with the light data values of the corresponding light control data sub-group is determined.

In certain embodiments, the correcting comprises dividing the light data values by the test data values for each test data sub-group and the corresponding light data sub-group.

In certain embodiments, the method further comprises converting the corrected test data values into coefficient values within a predetermined scale, and wherein the predetermined correlation data comprises different target analyte levels and their associated coefficient values of the at least one light parameter.

In certain embodiments, the test region is an elongate strip comprising a conjugate zone including conjugate particles which can react with the target analyte, at least one test band downstream of the conjugate zone, and a control band downstream of the at least one test line, wherein the at least one test band and the control band can produce a respective visual marker in the presence of the target analyte.

In certain embodiments, the at least one test band comprises a plurality of test bands, each test band of the plurality of test bands having a different or a same affinity for the target analyte.

In certain embodiments, the subdivisions of the test region are transverse to a longitudinal axis of the test strip.

In certain embodiments, the light control region is an elongate region parallel to the test region.

In certain embodiments, the subdivisions of the light control region are transverse to a longitudinal axis of the light control region.

In certain embodiments, a given subdivided area of the test strip has a corresponding subdivided area of the light control region, each pair of subdivided areas of the test strip and the light control region being aligned along an axis.

In certain embodiments, the light control region is white or black.

In certain embodiments, the method further comprises reducing a noise of the test data by: determining two reference points between subsequent peaks of the adjacent test data sub-groups; identifying a highest peak between the two reference points; and clipping the light intensity of the test data by an intensity equivalent to the highest peak.

In certain embodiments, the obtaining test data of a test region of a lateral flow device comprises: obtaining image data of a surface of the lateral flow device on which the test region is located; identifying at least one fiduciary marker on the image data, and determining the test region and the corresponding test data from the image data based on a predetermined location of the test region relative to the at least one fiduciary marker.

In certain embodiments, the method further comprises obtaining date data related to the lateral flow device and determining an ageing of the lateral flow assay, and retrieving from a database predetermined correlation data corresponding to the ageing of the lateral flow assay, and using the retrieved predetermined correlation data to determine the target analyte level.

In certain embodiments, the method further comprises determining a lapsed time between when the test was started on the lateral flow device and when the test data was captured.

In certain embodiments, the method further comprises obtaining identity data relating to the lateral flow device and sending the determined level of the target analyte of the test data to a recipient based on the identity data.

In certain embodiments, one or both of the date data and the identity data is obtained from a QR code on the lateral flow assay test.

From another aspect, there is provided a system for determining a level of a target analyte from a lateral flow device, the system comprising: a computing system configured to execute a method as described herein. The computing system may comprise a mobile device and include a camera for capturing one or more of the test data, the light control data and the color control data.

From yet another aspect, there is provided a lateral flow device comprising: a backing layer; an adhesive layer on a top surface of the backing layer; a top layer on the adhesive layer, the top layer including a cut-out region which is sized and shaped to receive a lateral flow assay test strip; and the lateral flow assay test strip positioned in the cut-out region and stuck to the backing layer by the adhesive layer.

In certain embodiments, the backing layer and the top layer have a same width and a same length, and optionally wherein the width and the length correspond to a size of a credit card. The backing layer and the top layer may be one piece and folded relative to each other.

In certain embodiments, a top surface of the top layer includes one or more of: a QR code, fiduciary markers, a light control region for compensating for differing ambient light intensity conditions; and a color control region for compensating for differing ambient light color conditions.

From a further aspect, there is provided a lateral flow system comprising a mobile smart device comprising a digital camera, a source of light and a processor, wherein the processor is configured to perform any of the method described herein. The lateral flow system may further comprise the lateral flow device as described herein and/or an electronic device for executing any of the method steps described herein.

In the context of the present specification, unless expressly provided otherwise, the expression “computer-readable medium” and “memory” are intended to include media of any nature and kind whatsoever, non-limiting examples of which include RAM, ROM, disks (CD-ROMs, DVDs, floppy disks, hard disk drives, etc.), USB keys, flash memory cards, solid state-drives, and tape drives.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

Embodiments of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of embodiments of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a block diagram of an example system comprising a lateral flow device and a computing system, in accordance with various embodiments of the present technology;

FIG. 2 is a top plan view of the lateral flow device of FIG. 1, in accordance with various embodiments of the present technology;

FIG. 3 is a cross-sectional view through the line A-A′ of FIG. 2, in accordance with various embodiments of the present technology;

FIG. 4 a top plan view of another lateral flow device, in accordance with different embodiments of the present technology;

FIG. 5 is a perspective view of yet another lateral flow device, in accordance with different embodiments of the present technology;

FIG. 6 is a block diagram of the computing system of FIG. 2, in accordance with various embodiments of the present technology;

FIG. 7 is a flow diagram of a method for determining a level of a target analyte from a lateral flow device, such as the lateral flow device of FIG. 1, in accordance with various embodiments of the present technology;

FIG. 8 is the lateral flow device of FIG. 2 showing a test band and a control band on a test strip, in accordance with various embodiments of the present technology;

FIG. 9 is a test region of the lateral flow device of FIG. 8 including at least a portion of the test strip of FIG. 8, in accordance with various embodiments of the present technology;

FIG. 10 is a light control region of the lateral flow device of FIG. 8, in accordance with various embodiments of the present technology;

FIG. 11A depicts example test data (upper image) of a light parameter from a test region (lower image) of a lateral flow device, in accordance with various embodiments of the present technology;

FIG. 11B depicts example light control data (upper image) of the light parameter from a light control region (lower image) of the lateral flow device, in accordance with various embodiments of the present technology;

FIG. 11C depicts the test data of FIG. 11A after correction with the light control data of FIG. 11B, in accordance with various embodiments of the present technology;

FIG. 12 is an example standard curve mapping different light intensities to different analyte levels;

FIG. 13 is a flow diagram of method steps additional to the method steps of FIG. 7, in accordance with various embodiments of the present technology;

FIG. 14 is a flow diagram of a method step additional to the method steps of FIG. 7, in accordance with various embodiments of the present technology; and

FIG. 15 is a flow diagram of a method step additional to the method steps of FIG. 7, in accordance with various embodiments of the present technology.

It should be noted that, unless otherwise explicitly specified herein, the drawings are not to scale.

### DETAILED DESCRIPTION

Certain aspects and embodiments of the present technology are directed to methods and systems for quantitative lateral flow tests.

### Systems

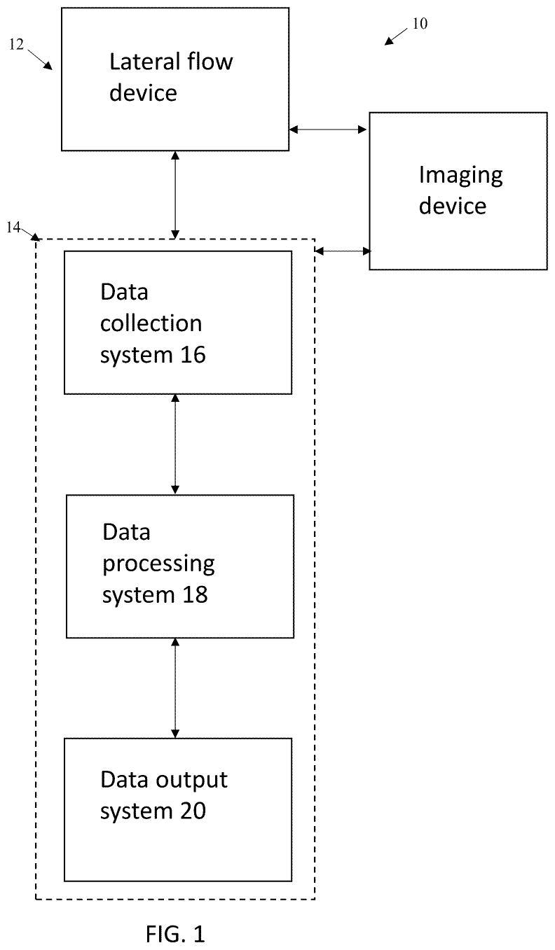

Referring to FIG. 1, there is a provided a system 10 for determining quantitative results from a lateral flow assay on a lateral flow device, according to embodiments of the present technology. The system 10 comprises a lateral flow device 12 for conducting a lateral flow assay, and at least one computing system 14 for imaging, processing and outputting quantitative results of the lateral flow test. The at least one computing system 14 may comprise a data collection system 16, a data processing system 18 and a data output system 20. Any one or more of the data collection system 16, the data processing system 18 and the data output system 20 may be embodied in the same or separate hardware, and may be sub-systems of the same or different computing system 14. The system 10 may include an imaging device 21, such a camera, for collecting data, which data can then be communicated to the computing system 14 and processed by the computer system 14. In other embodiments, any one or more of the data collection system 16, the data processing system 18 and the data output system 20 may be embodied with the imaging device, such as a smart phone.

It is to be expressly understood that the system 10 as depicted is merely an illustrative implementation of the present technology. Thus, the description thereof that follows is intended to be only a description of illustrative examples of the present technology. This description is not intended to define the scope or set forth the bounds of the present technology. In some cases, what are believed to be helpful examples of modifications to the system 10 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition, it is to be understood that the system 10 may provide in certain instances simple implementations of the present technology, and that where such is the case they have been presented in this manner as an aid to understanding. As persons skilled in the art would understand, various implementations of the present technology may be of a greater complexity.

### Lateral Flow Devices

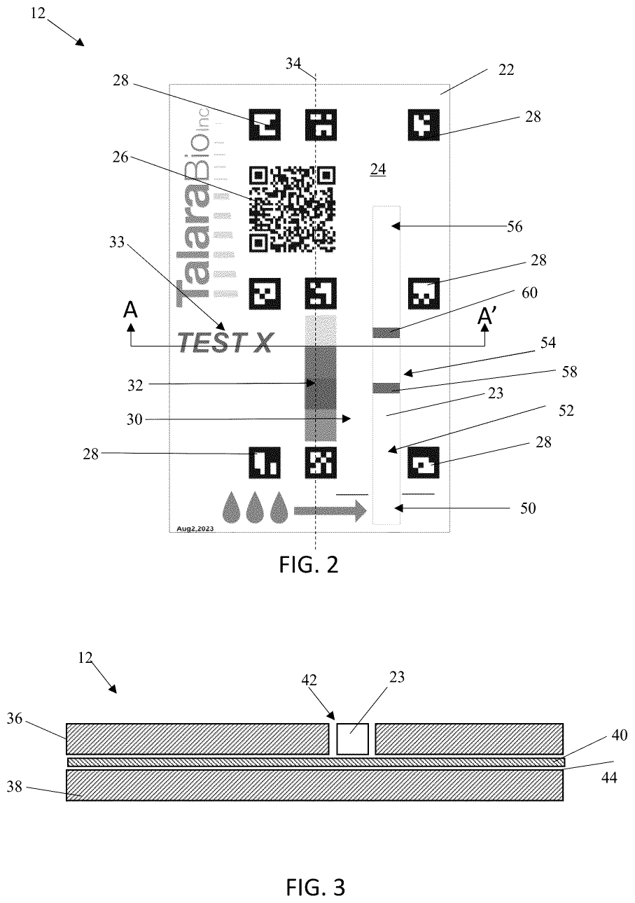

Turning to FIG. 2, there is shown an example lateral flow device 12 of the present technology.

The lateral flow device 12 comprises a body 22, also known as a cassette, supporting a test strip 23. The body 22 has a top surface 24 having certain markings thereon, including one or more of: a QR code 26, fiduciary markers 28, a light control region 30, which may be white, or any other color, for compensating for differing ambient light intensity conditions; a color control region 32 for compensating for differing ambient light color conditions; and reference information 33 such as information about the assay. The top surface 24 is planar. The light control region 30 may comprise a material made of the same material as the test strip 23.

The test strip 23 is elongate and is disposed parallel to a longitudinal axis 34 of the body 22 of the lateral flow device 12. The light control region 30 is elongate and is disposed on one side of the test strip 23. The color control region 32 is elongate and is disposed on another side of the test strip 23. Although the lateral flow device 12 of FIG. 2 shows a single light control region 30 and a single color control region 32, it will be appreciated that multiple light and color control regions may be provided. Furthermore, the placement and orientation of the light control region 30, color control region 32, test strip 23 may differ from that shown in the figures. The color control region 32 may be omitted in certain embodiments. As shown in FIG. 2, in certain embodiments, there are provided nine (9) fiduciary markers 28, but the lateral flow device 12 may have more than or less than nine fiduciary markers 28.

The top surface 24 may include any other markings, such as those required to comply with regulatory requirements. Example markings include, but are not limited to, hologram, security markings, quality status details (e.g. CE marking), instructions to the user, and the like.

As best see in FIG. 3, in certain embodiments, the body 22 has a layered construction and comprises a top layer 36 attached to a bottom layer 38 by adhesive 40. A cut-out 42 is defined in the top layer 36. The test strip 23 is stuck to the adhesive 40 and aligned with the cut-out 42 such that at least a portion of the test strip 23 can be accessed via the cut-out 42.

A method of manufacturing the lateral flow device 12 comprises providing the bottom layer 38, providing the adhesive 40 on a top surface 44 of the bottom layer 38, placing the top layer 36 on the adhesive 40, and placing the test strip 23 in the cut-out 42 of the top layer 36 in contact with the adhesive 40. The test strip 23 may be positioned on the adhesive 40 before or after the top layer 36 is positioned on the adhesive 40.

It will be appreciated, that the lateral flow device 12 may have any other construction. For example, in certain other embodiments, the lateral flow device 12 may have a single-body construction.

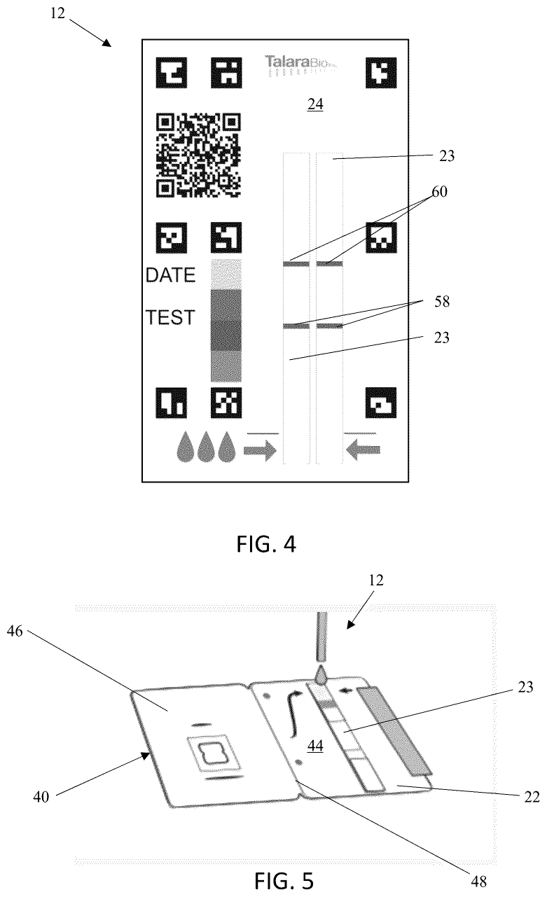

The lateral flow device 12 of FIG. 4 differs from that of FIG. 2 in that instead of there being provided a single test strip 23, two test strips 23 are provided. The two test strips 23 may have the same or different lateral flow assays thereon.

The lateral flow device 12 of FIG. 5 differs from that of FIG. 2 and FIG. 4 in that the body 22 includes a cover 46. The cover 46 is a flap foldably attached to the body 22 along one edge 48 so that it can be pivoted along the edge 48 to cover the top surface 44. This can help to keep contaminants off the test strip. Other configurations of covers 46 are within the scope of the present technology. It will be appreciated that the embodiments of the lateral flow device 12 of FIGS. 2 and 4 may also be provided with the cover 46.

In any of the embodiments of the lateral flow device 12, the body 22 may be of any suitable size or shape. In some examples, the body 22 is the shape of a credit card, enabling convenient handling and storage.

Turning now to the lateral flow assay and the test strip 23 of the lateral flow device 12, it will be appreciated that any suitable lateral flow assay may be used to detect the compound of interest, in a sample. In the example below, the lateral flow assay is configured to detect analytes as the compounds of interest. However, it will be appreciated that the present technology encompasses test strips 23 with other types of lateral flow assays that can detect compounds of interest other than analytes. The sample may comprise urine, blood, plasma, saliva or any other type of biological sample. The sample may comprise a biological sample in a buffer solution. The lateral flow assay may comprise a competitive or a sandwich assay. The lateral flow assay may be pre-applied to the test-strip 23.

In one example, the lateral flow assay comprises a monoclonal antibody specific in affinity for THC (delta-9-tetrahydrocannabinol) conjugated to 40 nm gold particles and deposited on the test strip 23. THC analyte is conjugated to BGG Bovine Gamma Globulin is deposited on the test strip 23. A control line of goat anti mouse antibody is also deposited on the test strip 23.

With reference to FIG. 2, in certain embodiments, the test strip 23 comprises a sample zone 50, a conjugate zone 52, a test zone 54 and a wicking zone 56. When the sample is introduced to the sample zone 50, such as in the form of drops of liquid, the sample will migrate to the conjugate zone 52 by virtue of capillary flow enabled by the wicking zone 56. The sample zone 50, the conjugate zone 52 and the test zone 54 are fluidly connected in a manner that supports capillary flow. In some embodiments, each of the zones 50, 52, 54, 56 comprise separate pads that are fluidly communicable. In some embodiments, the sample zone 50 may be omitted and the sample may be applied directly to the conjugate zone 52 or the test zone 54. In yet other embodiments, the conjugate zone 52 and the test zone 54 may be integral and comprise a single pad. Generally, the sample is loaded upstream from the test zone 54.

The conjugate zone 52 is coated or pre-loaded with conjugates which are analyte-recognizing molecules and including a label such as a colored particle label (e.g. gold). The conjugate zone 52 is configured to release the conjugates upon contact with the moving liquid sample. In the presence of the analytes, analyte-conjugate complexes form and will be carried by the liquid sample along the test strip 23 to the test zone 54. In the absence of the analytes, the unbound conjugates will flow along the test strip 23.

In certain embodiments, the analyte may be a protein, a polypeptide, a peptide, an antigen, an antibody, a hormone, an enzyme, a ligand, a polynucleotide, a small molecule, a carbohydrate, a drug, a metabolite or any other molecule that may be desirable to detect in a sample.

In certain embodiments, the analyte-recognizing molecule may be an antibody or an immunoglobulin derived or related molecule such an Fv, a scFv, a di-scFv, a Fab, a F (ab) 2, a nanobody (single-domain antibody) or other Camelidac derived molecule, an immunoglobulin new antigen receptor (IgNAR), a non-immunoglobulin-derived binding molecule such as an aptamer, an affimer, an avimer, a designed ankyrin repeated protein, a monobody, a proteincatalyzed capture agent (PCC), a knottin, an affinity clamp, an affibody molecule, a receptor or alternatively a ligand, an enzyme substrate, a polynucleotide of complementary sequence (when the target analyte is a polynucleotide sequence, DNA or RNA), or a protein.

As used herein, label includes a detectable indicator, including but not limited to labels which are soluble or particulate, metallic, organic or inorganic, and may include spectral labels such as green fluorescent protein, fluorescent dyes (for example fluorescein and its derivatives, rhodamine), chemiluminescent compounds (for example luciferin and luminol), spectral colorimetric labels such as colloidal gold, or carbon particles, colored glass or plastic (for example polystyrene, polypropylene, latex, etc.) beads. Where necessary or desirable, particle labels can be colored, for example by applying dye to particles. The term “colored particle label” includes but is not limited to, colored latex (polystyrene) particles, metallic (e.g., gold) sols, non-metallic elemental (e.g., selenium, carbon) sols and dye sols. In an embodiment of the disclosure detectable label may be gold nanoparticles, colored latex beads, and the like.

The test zone 54 includes immobilized antibodies that have an affinity for the analyte-conjugate complexes. Binding of the analyte-conjugate complexes with the immobilized antibodies will cause a visual change, such as a change in color due to the label of the analyte-conjugate complexes. The immobilized antibodies in the test zone 54 may be disposed as a band such that the color change causes a colored band (“test band 58”) to appear. In some embodiments, the immobilized antibodies may be disposed as multiple bands having different affinities for the analyte-conjugate complexes which can help in determining a quantitative amount of the analyte in the sample. For example, the multiple bands may have increasing affinity in the direction of fluid flow.

The test zone 54 also includes at least one band of immobilized secondary recognizing molecules configured to change in color in the presence of the analyte or the conjugate to show that the assay has worked (“control band 60”).

A visual intensity of the test band 58 may be a function of at least: (i) the concentration of the analyte in the sample; (ii) the affinities of the analyte for the analyte-recognizing molecule; and (iii) the amount of analyte-recognizing molecule molecules that bind each analyte.

The wicking zone 56 comprises a water-absorbing material or pad that acts as a waste liquid absorption zone.

As mentioned earlier, each of the sample, conjugate, test and a wicking zones 50, 52, 54, 56 may comprise a pad. Some of the pads of the different zones may be integral, or be individual pads and fluidly connectable to each other such as by touching or overlapping. One or more of the pads may be made of any material(s) suitable for permitting lateral flow of a fluid along the test strip. Lateral flow may refer to capillary flow through a material in a horizontal direction, and may apply also to the flow of a liquid from a point of application of the liquid to another lateral position even if, for example, the apparatus is held vertical or on an incline. Lateral flow depends upon properties of the liquid/substrate interaction (surface wetting or wicking action) and does not require or involve application of outside forces, e.g., vacuum or pressure applications by the user.

One or more of the pads may be made from materials, such as, cellulose fibers and various derivatives (e.g. nitrocellulose, nitrocellulose blends with polyester or cellulose, untreated paper, porous paper), rayon, glass fibers with or without binders, acrylonitrile copolymer or nylon or other porous materials that allow lateral flow.

### Computing System

### Computing Environment

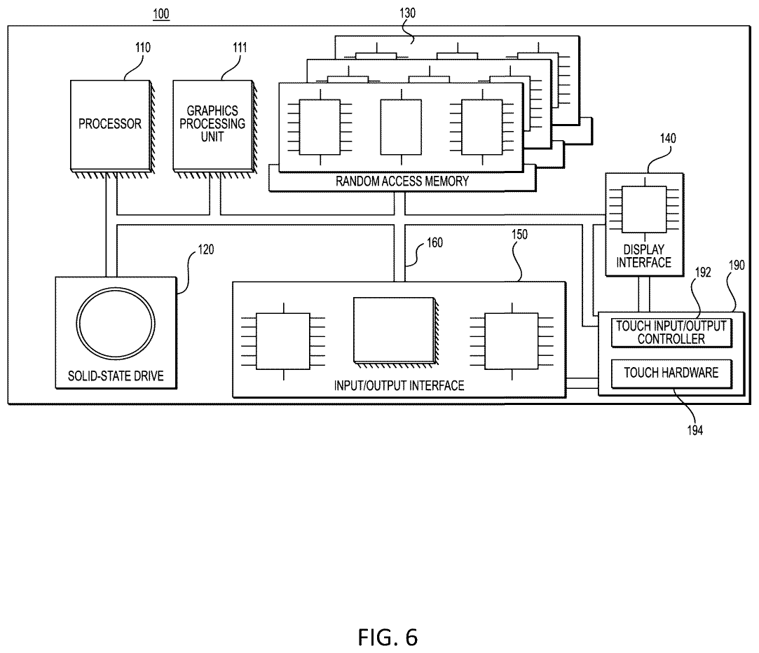

The computing system 14 is configured to determine quantitative test results from a lateral flow device and assay such as the lateral flow device 12 of any of FIGS. 2 to 5. As stated earlier, the computing system 14 may comprise the data collection system 16 for collecting data from the lateral flow device 12, a data processing system 18 for processing the collected data to determine quantitative results and a data output system 20 for outputting the determined quantitative results. Turning now to FIG. 6, the computing system 14 has a computing environment 100, which may be used to implement and/or execute any of the methods described herein.

Any one or more of the data collection system 16, the data processing system 18 and the data output system 20 may be embodied in the same or separate computing environment 100. Any one or more of the data collection system 16, the data processing system 18 and the data output system 20 may be sub-systems of the same or different computing system 14.

In some embodiments, the computing environment 100 may be implemented by any of a conventional personal computer, a network device and/or an electronic device (such as, but not limited to, a mobile device, a tablet device, a server, a controller unit, a control device, etc.), and/or any combination thereof appropriate to the relevant task at hand. In some embodiments, the computing environment 100 is implemented in a smart phone having a camera, and optionally a flash.

In some embodiments, the computing environment 100 comprises various hardware components including one or more single or multi-core processors collectively represented by processor 110, a solid-state drive 120, a random access memory 130, and an input/output interface 150. The computing environment 100 may be a computer specifically designed to operate a machine learning algorithm (MLA). The computing environment 100 may be a generic computer system.

In some other embodiments, the computing environment 100 may be an “off-the-shelf” generic computer system. In some embodiments, the computing environment 100 may also be distributed amongst multiple systems. The computing environment 100 may also be specifically dedicated to the implementation of the present technology. As a person in the art of the present technology may appreciate, multiple variations as to how the computing environment 100 is implemented may be envisioned without departing from the scope of the present technology.

Those skilled in the art will appreciate that processor 110 is generally representative of a processing capability. In some embodiments, in place of or in addition to one or more conventional Central Processing Units (CPUs), one or more specialized processing cores may be provided. For example, one or more Graphic Processing Units 111 (GPUs), Tensor Processing Units (TPUs), and/or other so-called accelerated processors (or processing accelerators) may be provided in addition to or in place of one or more CPUs.

System memory will typically include random access memory 130, but is more generally intended to encompass any type of non-transitory system memory such as static random access memory (SRAM), dynamic random access memory (DRAM), synchronous DRAM (SDRAM), read-only memory (ROM), or a combination thereof. Solid-state drive 120 is shown as an example of a mass storage device, but more generally such mass storage may comprise any type of non-transitory storage device configured to store data, programs, and other information, and to make the data, programs, and other information accessible via a system bus 160. For example, mass storage may comprise one or more of a solid state drive, hard disk drive, a magnetic disk drive, and/or an optical disk drive.

Communication between the various components of the computing environment 100 may be enabled by a system bus 160 comprising one or more internal and/or external buses (e.g., a PCI bus, universal serial bus, IEEE 1394 “Firewire” bus, SCSI bus, Serial-ATA bus, ARINC bus, etc.), to which the various hardware components are electronically coupled.

The input/output interface 150 may allow enabling networking capabilities such as wired or wireless access. As an example, the input/output interface 150 may comprise a networking interface such as, but not limited to, a network port, a network socket, a network interface controller and the like. Multiple examples of how the networking interface may be implemented will become apparent to the person skilled in the art of the present technology. For example the networking interface may implement specific physical layer and data link layer standards such as Ethernet, Fibre Channel, Wi-Fi, Token Ring or Serial communication protocols. The specific physical layer and the data link layer may provide a base for a full network protocol stack, allowing communication among small groups of computers on the same local area network (LAN) and large-scale network communications through routable protocols, such as Internet Protocol (IP).

The input/output interface 150 may be coupled to a touchscreen 190 and/or to the one or more internal and/or external buses 160. The touchscreen 190 may be part of the display. In some embodiments, the touchscreen 190 is the display. The touchscreen 190 may equally be referred to as a screen 190. In the embodiments illustrated in FIG. 6, the touchscreen 190 comprises touch hardware 194 (e.g., pressure-sensitive cells embedded in a layer of a display allowing detection of a physical interaction between a user and the display) and a touch input/output controller 192 allowing communication with the display interface 140 and/or the one or more internal and/or external buses 160. In some embodiments, the input/output interface 150 may be connected to a keyboard (not shown), a mouse (not shown) or a trackpad (not shown) allowing the user to interact with the computing environment 100 in addition to or instead of the touchscreen 190.

According to some implementations of the present technology, the solid-state drive 120 stores program instructions suitable for being loaded into the random access memory 130 and executed by the processor 110 for executing acts of one or more methods described herein. For example, at least some of the program instructions may be part of a library or an application.

As stated earlier, the computing system 14 may include a data collection system 16 for collecting data from the lateral flow device 12, which may include data relating to the lateral flow assay on the test strip 23 as well as data about the top surface 24 of the cassette. The data may comprise image data, such as one or more of image data of information on the top surface 24 of the lateral flow device 12 (e.g. QR code, reference label, etc), light parameter data of light reflected from the test strip 23, and light parameter data from a reference area on the top surface 24 of the lateral flow device 12. The data collected by the data collection system 16 may have any format.

In certain embodiments, the data includes one or more of: (i) image data of at least a portion of the test strip 23 before, during or at the conclusion of the lateral test assay; (ii) light intensity/hue/saturation data of at least a portion of the test strip 23; (iii) light color data of at least a portion of the test strip 23; (iv) image data of one or more of the fiduciary markers 28 on the top surface 24 of the cassette, (v) image data of the light control region 30, (vi) light intensity/hue/saturation data of light reflected from the light control region 30; (vii) color intensity/hue/saturation data of light reflected from the light control region 30; (viii) image data of the color control region 32, (ix) light intensity/hue/saturation data of light reflected from the color control region 32; (x) color intensity data of light reflected from the color control region 32; (xi) image data of the QR code 26, (xii) image data of the reference information 33, (xiii) image data of the fiduciary markers 28.

The data collection system 16 may be configured to collect the data directly from the lateral flow device 12, or to receive stored data from another source such as the mass storage device 120 of the computing system 14 or of another computing system. The data collection system 16 comprises, in certain embodiments, a camera such as a camera of a mobile device. The camera may include a light source such as a flash. The data collection system 16 may include a timing function for providing time stamps, etc.

The data processing system 18 is configured to use any of the data, with or without additional data, to execute one or more of the methods described and/or claimed herein in order to determine a quantitative test result of the lateral flow assay of the lateral flow device 12. The quantitative test result may be in the form of a numerical concentration of the compound of interest (analyte) in the sample, a concentration range, a visual indicator/sound/haptic signal representative of the concentration of the compound of interest.

The data output system is configured to cause the determined quantitative test result to be output, such as to a display device, or to be uploaded to a database, or to be sent over a network.

### Methods

Certain aspects and embodiments of the present technology comprise a method 300 to obtain quantitative lateral test results from a lateral flow device 12. The methods 30 described herein may be fully or at least partially automated so as to minimize an input of a user.

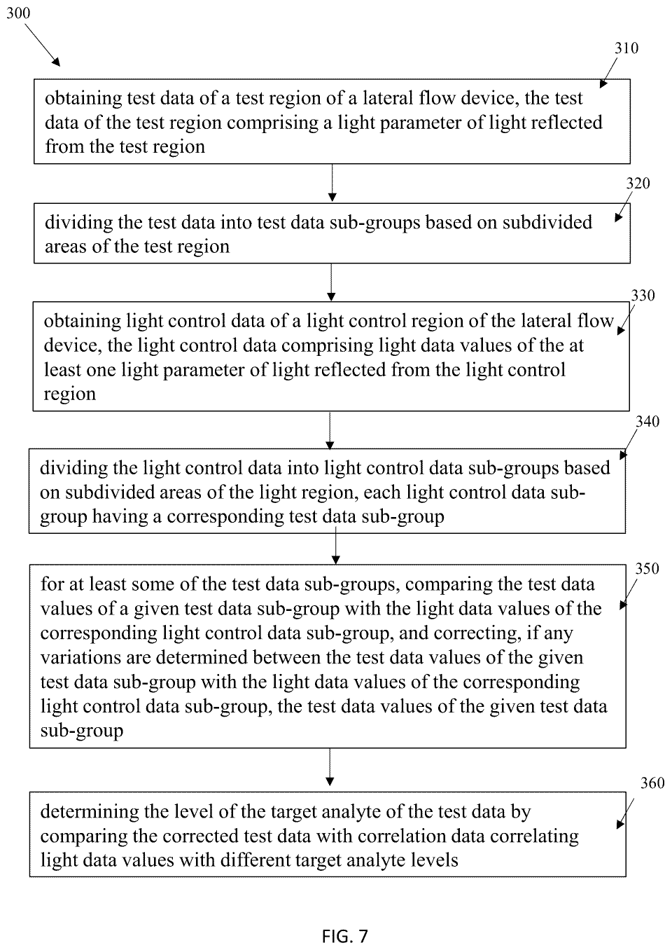

FIG. 7 is a flow diagram of certain aspects of the method 300 for determining a level of a compound of interest from a lateral flow device, such as the lateral flow device 12. All or portions of the method 300 may be executed by one or more of the computing system 14, data collection system 16, data processing system 18, and/or the data output system 20. In one or more aspects, the method 300 or one or more steps thereof may be performed by the computing system including the computing environment 100. The method 300 or one or more steps thereof may be embodied in computer-executable instructions that are stored in a computer-readable medium, such as a non-transitory mass storage device 120, loaded into memory and executed by a CPU. The method 300 is exemplary, and it should be understood that some steps or portions of steps in the flow diagram may be omitted and/or changed in order.

Step 310: Obtaining Test Data of a Test Region of a Lateral Flow Device, the Test Data of the Test Region Comprising a Light Parameter Reflected from the Test Region

At step 310, the method 300 comprises obtaining test data of a test region of a lateral flow device, the test data of the test region comprising a light parameter reflected from the test region.

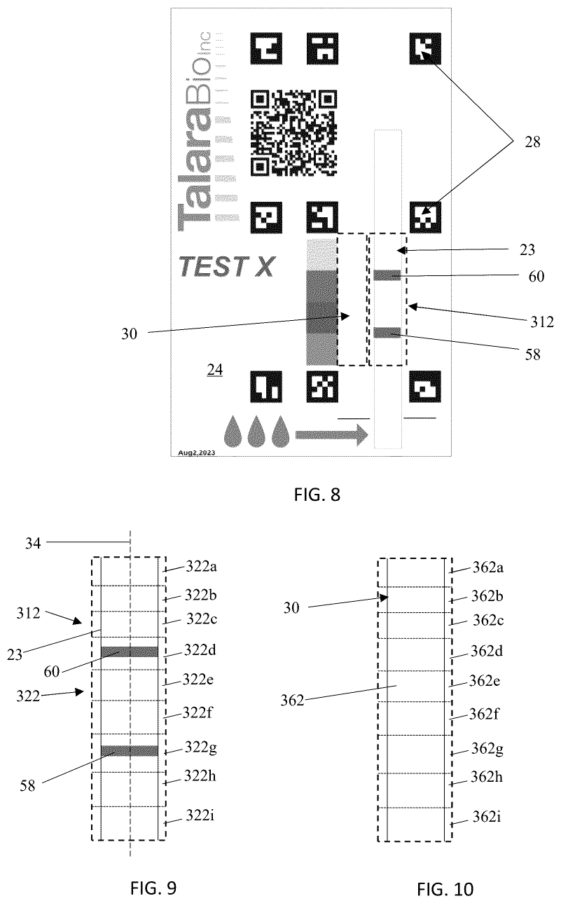

The lateral flow device may comprise the lateral flow device 12 described herein and the test region may comprise the test region 312 shown in FIG. 8 which includes at least portion of the test strip 23 of the lateral flow device 12. The test region 312 includes at least the portion of the test strip 23 with the test band 58 and the control band 60.

Obtaining the test data of the test region 312 may comprise obtaining image data of the entire top surface 24 of the lateral flow device 12, then determining the test region 312 using the fiduciary markers 28. The location of the test region 312 relative to the fiduciary markers 28 may be predetermined. The test data may then be obtained by extracting data associated with the determined test region from data of the entire top surface 24.

In certain embodiments, the test data includes light parameters of light reflected from the test region 312. The light parameters may include any one or more of light intensity, light hue, light saturation and light wavelength. Other light parameters are within the scope of the present technology.

In some embodiments, the test data is a light wavelength-intensity distribution (wavelength along the x axis and intensity along the y axis).

The test data may have been collected responsive to a light flash, such as a camera flash.

Step 320: Dividing the Test Data into Test Data Sub-Groups Based on Subdivided Areas of the Test Region

At step 320, the method 300 comprises dividing the test data into test data sub-groups based on subdivisions of the test region. In certain embodiments, such as the embodiment shown in FIG. 9, the subdivisions of the test region 312 are lateral bands 322 (322a, 322b, 322c, 322d, 322e, 322f, 322g, 322h, 322i) that are transverse to the longitudinal axis 34. The test data sub-groups thus comprise the test data divided as per the subdivisions of the test region 312.

In other embodiments, the test region 312 and its associated data may be subdivided in any other manner, such as longitudinal sub-divisions instead of transverse sub-divisions or grid subdivisions.

Any number of sub-divisions are within the scope of the present technology. In some embodiments, there are up to 2000 lateral sub-divisions. In other embodiments, there are between 500-1500 sub-divisions. In some embodiments, Step 320 may be omitted and the test data may be treated without sub-dividing.

The method 300 may further comprise filtering the data within each test data sub-group according to predetermined rules. This may help to reduce or eliminate noise in the data. For example, light intensity values above or below predetermined threshold levels may be discarded. Other filtering steps are within the scope of the present technology such as normalizing relative to a highest peak value or a lowest peak value, snipping data points between peaks, etc. In one filtering method, two reference points are identified between adjacent peaks of adjacent test data sub-groups, a highest peak is identified between the two reference points, the light intensity of the test data is modulated by an intensity equivalent to the highest peak.

Step 330: Obtaining Light Control Data of a Light Control Region of the Lateral Flow Device, the Light Control Data Comprising Light Data Values of the at Least One Light Parameter of Light Reflected from the Light Control Region

In certain embodiments, the method 300 comprises obtaining light control data of a light control region of the lateral flow device, such as the light control region 30 of the lateral flow device 12. The light control data comprises a light parameter of light reflected from the light control region.

Obtaining light control data of the light control region 30 of the test region 312 may comprise obtaining image data of the entire top surface 24 of the lateral flow device 12, then determining the light control region 30 using the fiduciary markers 28. The location of the light control region 30 relative to the fiduciary markers 28 may be predetermined. The light control data may then be obtained by extracting light parameter data associated with the determined light control region 30. The exact location of the light control region 30 is not important as long as the light control region 30 is sufficiently close to the test strip 23 so as to be subject to similar ambient light conditions as the test strip 23.

In certain embodiments, the light control data includes light parameters of light reflected from the light control region 30. The light parameters may include any one or more of light intensity, light hue, light saturation and light wavelength. Other light parameters are within the scope of the present technology. The light parameter of the light control data is the same light control parameter of the test data.

In some embodiments, the light control data is a light wavelength-intensity distribution (wavelength along the x axis and intensity along the y axis).

The light control data may have been collected responsive to a light flash, such as a camera flash.

The light control data may have been collected at the same time as the test data.

Step 340: Dividing the Light Control Data into Light Control Data Sub-Groups Based on Subdivided Areas of the Light Region, Each Light Control Data Sub-Group Having a Corresponding Test Data Sub-Group

The method 300 may then comprise dividing the light control data into light control data sub-groups, the subdivided areas of the light control region 30 corresponding to the subdivided areas of the test region. As seen in FIG. 10, in some embodiments, the light control data sub-groups are lateral bands which are transverse to a longitudinal axis of the light control region. The light control data sub-groups thus comprise the light control data divided as per the subdivisions of the light control region 30. Each subdivided area of the test region has a corresponding subdivided area of the light control region. They can be considered as pairs, such as 322a-362a, 322b-362b, 322c-362c, etc. In certain embodiments, the pairs are adjacent to one another or proximate one another.

In other embodiments, the light control region 30 and its associated data may be subdivided in any other manner, such as longitudinal sub-divisions instead of transverse sub-divisions or grid subdivisions.

Any number of sub-divisions are within the scope of the present technology. In some embodiments, there are up to 2000 lateral sub-divisions. In other embodiments, there are between 500-1500 sub-divisions. In some embodiments, Step 340 may be omitted and the light control data may be treated without sub-dividing.

Step 350: For at Least Some of the Test Data Sub-Groups, Comparing the Test Data Values of a Given Test Data Sub-Group with the Light Data Values of the Corresponding Light Control Data Sub-Group, and Correcting, if any Variations are Determined Between the Test Data Values of the Given Test Data Sub-Group with the Light Data Values of the Corresponding Light Control Data Sub-Group, the Test Data Values of the Given Test Data Sub-Group

The method 300 then comprises, for at least some of the test data sub-groups, comparing the light parameter of the test data sub-group 322 with the corresponding light parameter of the light control data sub-group of the light control region 30, and correcting the light parameter of each test data sub-group. In certain embodiments, all of the test data sub-groups are taken into account for this step. In other embodiments, only a portion of the test data sub-groups are taken into account, such as those sub-groups including the test band and/or the control band (such as 322g and 322d in FIG. 9).

The effect of this correction is more accurate results based on correcting for ambient light conditions. For example, if there is a shadow across a portion of the light control region 30, it is assumed that the corresponding portion of the test region 312 will also have the shadow. Therefore, the effect of the shadow on the light parameter can be identified, and the light parameter values of the test region 312 can be corrected accordingly. The correction made may be proportional to the detected difference between the light parameters of test data sub-group pairs between the test region 312 and the light control region 30.

In some embodiments, the correcting comprises adjusting the test data values only of the given test data sub-group for which variation with the light data values of the corresponding light control data sub-group is determined. In other embodiments, all the test data values of all the given test data sub-groups are corrected.

In embodiments in which the light parameter is an intensity of the light of different wavelengths reflected from the test region 312, as reflected light intensity is lower from darker regions than light regions due to light absorption by darker colors, it will be appreciated that the data for test data sub-groups which include the test band 58 or the control band 60 will have lower values than those sub-groups which do not include the test or control bands 58, 60. The light intensity test data may resemble a bell curve. As the method 300 is concerned with quantifying the concentration of the compound of interest through an intensity of the test band 58, in some embodiments, the light parameter data may be inverted so that the areas of least light reflection show the higher peaks of light reflection. In such embodiments, determining a peak value comprises determining a highest value of the inverted sub-groups. Inversion is optional.

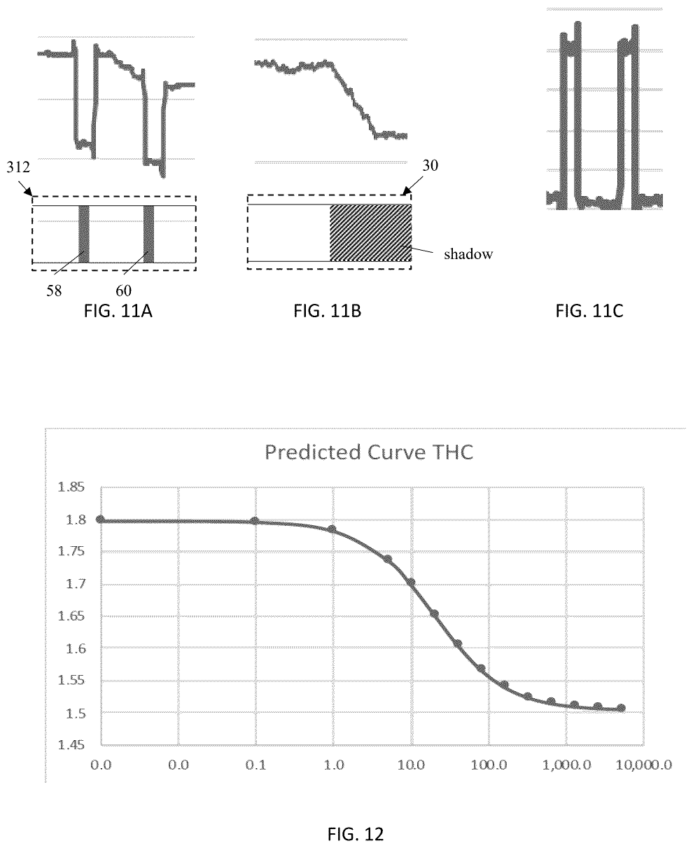

FIGS. 11A, 11B and 11C show one example of the correction. In FIG. 11A, the obtained reflected light intensity across the test region 312 is shown. The inverted peaks correspond to the light reflected from the test and control bands 58, 60. FIG. 11B shows the reflected light intensity across the light control region 30. As the light control region does not have any test and/or control bands, there are no peaks. However, as can be seen, the light intensity is lower in some parts of the light control region 30 than others and this may be attributed to a shadow, for example. FIG. 11C shows the test data after correcting for the shadow, and inverting the peaks.

In some embodiments, the correcting comprises dividing the light data values by the test data values for each of the test data sub-groups and its corresponding light data sub-group. In such cases, the correction also inverts the peaks. In other embodiments, the correcting comprises dividing the test data values by the light data values, and inverting in any other manner.

In other embodiments, the correcting can be performed in any other manner. For example, the light control region 30 may include a light reference band (not shown) with a predetermined light parameter value. By detecting the actual light parameter value of the light reference band in the light control region, it can be determined whether a correction is needed. If so, the correction can be made according to the actual light parameter value relative to the predetermined light parameter. The correction made may be proportional to the detected difference.

In other embodiments, the correcting comprises, for each test data sub-group, determining a representative value of the test data sub-group (e.g. peak, average, mean, median, etc) and comparing it with a representative value (e.g. peak, average, mean, median, etc) of the corresponding light control data sub-group. peak value of the light parameter.

In other embodiments in which the data is not inverted, determining the peak value comprises determining a lowest value of the test data sub-group.

Step 360: Determining the Level of the Target Analyte of the Test Data by Comparing the Corrected Test Data with Correlation Data Correlating Light Data Values with Different Target Analyte Levels

At step 360, the method 300 comprises determining the level of the target analyte of the test data by comparing the corrected test data with correlation data that correlates light data values of the given light parameter with different target analyte levels.

In some embodiments, coefficient values are derived from the corrected test data and compared with coefficient values of the correlation data. This is essentially a standardization step. The coefficient value scale may comprise any suitable scale such as 0 to 100, 0 to 50, 0 to 10, or the like.

The correlation may comprise a standard test curve correlating different target analyte levels with the corrected test data or with the coefficients derived from the corrected test data. FIG. 12 depicts an example standard test curve of the lateral flow device 12 for detecting THC.

The standard test curve may be predetermined by mapping different concentrations of the analyte against the light parameter such as light intensity. This may have been performed in manners known in the art, such as a four parameter logistic regression at concentrations of 0, 1, 10, 100 of analyte.

### Ageing Correction

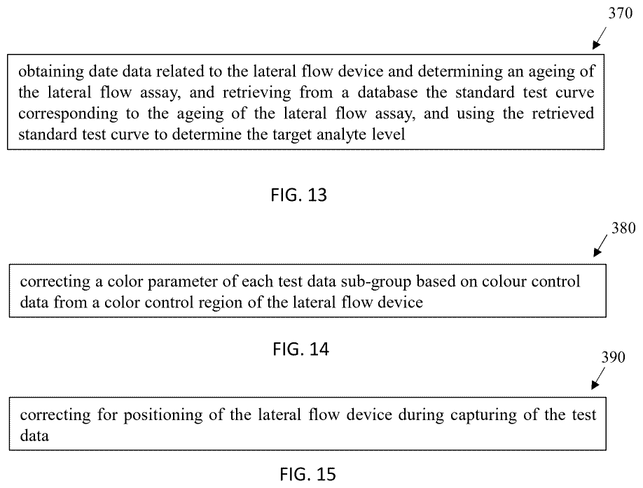

In certain embodiments, the method 300 includes a step for ageing correction (FIG. 13).

Step 370: Obtaining Date Data Related to the Lateral Flow Device and Determining an Ageing of the Lateral Flow Assay, and Retrieving from a Database the Standard Test Curve Corresponding to the Ageing of the Lateral Flow Assay, and Using the Retrieved Standard Test Curve to Determine the Target Analyte Level.

In certain embodiments, in order to account for a variation in a precision of the lateral flow assay over time (i.e. ageing), the method 300 includes step 370 for identifying any ageing effects and correcting for the ageing. The ageing effect may be observed between a manufacture time and a use time of the lateral flow device including the lateral flow assay. The manufacture time may be included in the reference information 33 and/or the QR code 26. The ageing effect may be observed between an imaging time of the test region and a start time of the lateral flow assay.

For example, in some embodiments, a difference in percent coefficient of variation (CV) between an imaging time of the test region and a start time of the lateral flow assay was determined: CV % is about 0.56% if the test region 312 is imaged between 15 to 30 minutes from a start time of the lateral flow assay compared to about 2.5% if the test region 312 is imaged between 30 minutes to 5 days from a start time of the lateral flow assay.

### Color Correction

In certain embodiments, the method 300 includes a step for color correction (FIG. 14).

Step 380: Correcting a Color Parameter of Each Test Data Sub-Group Based on Color Control Data from a Color Control Region of the Lateral Flow Device

In certain embodiments, the method 300 comprises obtaining color control data of a color control region of the lateral flow device, such as the color control region 32 of the lateral flow device 12. The color control data comprises a color parameter of light reflected from the color control region.

Obtaining color control data of the color control region 32 of the test region 312 may comprise obtaining image data of the entire top surface 24 of the lateral flow device 12, then determining the color control region 32 using the fiduciary markers 28. The location of the color control region 32 relative to the fiduciary markers 28 may be predetermined. The color control data then may be obtained by extracting color data associated with the determined color control region 30.

The control data may have been collected responsive to a light flash, such as a camera flash.

The color control data may have been collected at the same time as the test data and/or the light control data.

The method 300 may then comprise dividing the color control data into color control data sub-groups, by subdividing the color control region 32. The subdivisions of the color control region 32 may correspond to the subdivisions of the test region. The method 300 then comprises, for each subdivision of the color control region 32, comparing the color parameter of the test data sub-group 322 with the corresponding color parameter of the color control data sub-group of the color control region 32, and correcting the color parameter of each test data sub-group.

### Positioning Correction

In certain embodiments, the method 300 includes a step for correction of a positioning of the lateral flow device 12 during imaging, such as skew and yaw (FIG. 15).

### Step 390: Correcting for Positioning of the Lateral Flow Device During Capturing of the Test Data

In certain embodiments, step 390 comprises imaging at least a portion of the top surface 24 of the lateral flow device 12 including at least some of the fiduciary markers 28; comparing the imaged position of the fiduciary markers 28 with a predetermined position of the fiduciary markers 28; and determining differences between the imaged position of the fiduciary markers 28 with a predetermined position of the fiduciary markers 28. The geometric transformation needed to align the imaged and predetermined positions is identified and performed. Geometric transformations may include one or more of rotation, scaling and 4 point image transformation.

Step 390 may be performed before the light and color corrections described above.

After applying the transformation, the image may be further processed to identify any artifacts or misalignments between the fiduciary markers 24 as images and those that a predetermined. Additional adjustments are made if the fiducials are no longer visible or misaligned.

### Outputs

The method 300 may further comprise outputting the level of the determined target analyte by causing the determined target analyte level to be displayed on a display.

The method 300 may further comprise outputting the level of the determined target analyte by causing a device to emit an alert, such as a sound, haptic or visual signal. The device may comprise a mobile device of the user such as a smartphone.

The method 300 may comprise sending the determined target analyte level over a network to a computing system, which may be the same or different than the computing system 14. For example, the determined quantitative target analyte levels may be sent to the patient, or to the patient's doctor. The recipient of the determined target analyte level may be identified by imaging the reference information 33 on the top surface 24 of the lateral flow device 12. The reference information 33 may include one or more of information relating to the recipient, and information relating to the patient.

In certain embodiments, the method 300 comprises storing the determined quantitative target analyte level.

The method 300 may further comprise using the determined target analyte level to make diagnostic predictions.

While some of the above-described implementations may have been described and shown with reference to particular acts performed in a particular order, it will be understood that these acts may be combined, sub-divided, or re-ordered without departing from the teachings of the present technology. At least some of the acts may be executed in parallel or in series. Accordingly, the order and grouping of the act is not a limitation of the present technology.

It should be expressly understood that not all technical effects mentioned herein need be enjoyed in each and every embodiment of the present technology.

As used herein, the wording “and/or” is intended to represent an inclusive-or; for example, “X and/or Y” is intended to mean X or Y or both. As a further example, “X, Y, and/or Z” is intended to mean X or Y or Z or any combination thereof.

The foregoing description is intended to be exemplary rather than limiting. Modifications and improvements to the above-described implementations of the present technology may be apparent to those skilled in the art.

1. A method for determining a level of a target analyte in a sample from a lateral flow device, the method being executed by a processor of a computer system, the method comprising:
obtaining test data of a test region of a lateral flow device to which the sample has been applied, the test data of the test region comprising test data values of at least one light parameter of light reflected from the test region;
dividing the test data into test data sub-groups based on subdivided areas of the test region;
obtaining light control data of a light control region of the lateral flow device, the light control data comprising light data values of the at least one light parameter of light reflected from the light control region;
dividing the light control data into light control data sub-groups based on subdivided areas of the light control region, each light control data sub-group having a corresponding test data sub-group;
for at least some of the test data sub-groups,
comparing the test data values of a given test data sub-group with the light data values of the corresponding light control data sub-group, and
correcting, if any variations are determined between the test data values of the given test data sub-group with the light data values of the corresponding light control data sub-group, the test data values of the given test data sub-group; and
determining the level of the target analyte of the test data by comparing the corrected test data with predetermined correlation data correlating light data values with different target analyte levels.

2. The method of claim 1, wherein the correcting comprises adjusting the test data values only of the given test data sub-group for which variation with the light data values of the corresponding light control data sub-group is determined.

3. The method of claim 1, wherein the correcting comprises dividing the light data values by the test data values for each test data sub-group and the corresponding light data sub-group, or the correcting comprises dividing the test data values by the light data values for each test data sub-group and the corresponding light data sub-group.

4. The method of claim 1, further comprising converting the corrected test data values into coefficient values within a predetermined scale, and wherein the predetermined correlation data comprises different target analyte levels and their associated coefficient values of the at least one light parameter.

5. The method of claim 1, wherein the test region is an elongate strip comprising a conjugate zone including conjugate particles which can react with the target analyte, at least one test band downstream of the conjugate zone, and a control band downstream of the at least one test line, wherein the at least one test band and the control band can produce a respective visual marker in the presence of the target analyte.

6. The method of claim 5, wherein the at least one test band comprises a plurality of test bands, each test band of the plurality of test bands having a different or a same affinity for the target analyte.

7. The method of claim 1, wherein the subdivisions of the test region are transverse to a longitudinal axis of the test strip.

8. The method of claim 7, wherein the light control region is an elongate region parallel to the test region.

9. The method of claim 8, wherein the subdivisions of the light control region are transverse to a longitudinal axis of the light control region.

10. The method of claim 1, wherein a given subdivided area of the test strip has a corresponding subdivided area of the light control region, each pair of subdivided areas of the test strip and the light control region being aligned along an axis.

11. The method of claim 1, wherein the light control region is white or black.

12. The method of claim 1, further comprising reducing a noise of the test data by:
determining two reference points between subsequent peaks of the adjacent test data sub-groups;
identifying a highest peak between the two reference points; and
clipping the light intensity of the test data by an intensity equivalent to the highest peak.

13. The method of claim 1, wherein the obtaining test data of a test region of a lateral flow device comprises:
obtaining image data of a surface of the lateral flow device on which the test region is located;
identifying at least one fiduciary marker on the image data, and
determining the test region and the corresponding test data from the image data based on a predetermined location of the test region relative to the at least one fiduciary marker.

14. The method of claim 1, further comprising obtaining date data related to the lateral flow device and determining an ageing of the lateral flow assay, and
retrieving from a database predetermined correlation data corresponding to the ageing of the lateral flow assay, and using the retrieved predetermined correlation data to determine the target analyte level.

15. The method of claim 1, further comprising determining a lapsed time between when the test was started on the lateral flow device and when the test data was captured.

16. The method of claim 1, obtaining identity data relating to the lateral flow device and sending the determined level of the target analyte of the test data to a recipient based on the identity data.

17. The method of claim 16, wherein one or both of the date data and the identity data is obtained from a QR code on the lateral flow assay test.

18. A system for determining a level of a target analyte from a lateral flow device, the system comprising:
a computing system configured to execute a method as described in claim 1.

19. The system of claim 18, wherein the computing system comprises a mobile device and includes a camera for capturing one or more of the test data, the light control data and the color control data.

20. A lateral flow device comprising:
a backing layer,
an adhesive layer on a top surface of the backing layer,
a top layer on the adhesive layer, the top layer including a cut-out region which is sized and shaped to receive a lateral flow assay test strip; and
the lateral flow assay test strip positioned in the cut-out region and stuck to the backing layer by the adhesive layer.
